(19) United States
(12) Patent Application Publication
(10) Pub. No.: US 2009/0155369 A1
(43) Pub. Date: Jun. 18, 2009

(54) PHARMACEUTICAL COMPOSITION CONTAINING LEVODOPA, ENTACAPONE AND CARBIDOPA

(75) Inventors: Marta Huguet Riba, Barcelona (ES); Ignacio Diez Martin, Barcelona (ES)

Correspondence Address: MOORE & VAN ALLEN PLLC, P.O. BOX 13706, Research Triangle Park, NC, 27709, US

(73) Assignee: LABORATORIOS LESVI, S.L., Barcelona (ES)

(21) Appl. No.: 12/112,761

(22) Filed: Apr. 30, 2008

(30) Foreign Application Priority Data
Dec. 13, 2007 (EP) 07380354.6

(51) Int. Cl. A61K 9/14; A61K 31/195

(52) U.S. Cl. 424/490; 514/567

(57) ABSTRACT

The present invention refers to a solid pharmaceutical composition of entacapone, levodopa and carbidopa or pharmaceutically acceptable salts thereof characterized in that entacapone is in the form of granules and it is added separately to levodopa and carbidopa. In addition, this invention provides the process for its preparation.

### CROSS REFERENCE TO RELATED APPLICATIONS

This application claims priority to European Patent Application No. EP07380354.6, filed Dec. 13, 2007 and entitled “Pharmaceutical Composition Containing Levodopa, Entacapone and Carbidopa” in the name of Marta Huguet Riba et al., incorporated herein by reference in its entirety.

### FIELD OF THE INVENTION

The present invention relates to a new pharmaceutical composition of entacapone, levodopa and carbidopa or pharmaceutically acceptable salts or hydrates thereof characterized in that entacapone or a pharmaceutically acceptable salt thereof is in the form of granules and it is added separately to levodopa and carbidopa. The invention also relates to a preparation method of this composition.

### BACKGROUND OF THE INVENTION

Entacapone or (E)-2-cyano-3-(3,4-dihydroxy-5-nitrophenyl)-N,N-diethyl-2-propenamide is described in U.S. Pat. No. 5,446,194 as a cathecol-O-methyltransferase (COMT) inhibitor. An oral pharmaceutical composition containing entacapone and crosscarmelose sodium is marketed in Europe as COMTESS® and COMTAN®. These products are indicated as an adjunct to standard preparations of levodopa/benserazide or levodopa/carbidopa for use in patients with Parkinson's disease and end-of-dose motor fluctuations who cannot be stabilized on those combinations.

Levodopa and carbidopa are commonly used drugs in the treatment of Parkinson's disease. Combination of levodopa and carbidopa in form of tablets is marketed under the trademark SINEMET®.

Combination of levodopa, carbidopa and entacapone is marketed under the trademark STALEVO® to Orion and it is indicated for the treatment of patients with Parkinson's disease and end-of-dose motor fluctuations not stabilized on levodopa/dopa decarboxylase (DDC) inhibitor treatment.

European patent EP 1 189 608 B1 describes a pharmaceutical composition comprising entacapone, levodopa and carbidopa or pharmaceutically acceptable salts or hydrates thereof wherein carbidopa is added separately to the composition, e.g. entacapone and levodopa, together with (an) excipient(s). The patent indicates that, commonly used excipients (microcrystalline cellulose, and/or surface active agents such as polyethylene glycol, polysorbate and sodium lauryl sulphate and/or silica) are not suitable to be used in the formulation because they are incompatible with the drug combination. Moreover, formulations where all three ingredients are wet granulated together provided insufficient absorption of carbidopa.

Therefore, there is still a need for developing pharmaceutical compositions which incorporate levodopa, carbidopa and entacapone or their pharmaceutical acceptable salts and methods for preparing such compositions with an improved physical stability and without the active ingredient release properties being affected.

### BRIEF DESCRIPTION OF THE INVENTION

The aim of the present invention is to provide pharmaceutical compositions containing levodopa, carbidopa and entacapone or a pharmaceutically acceptable salt thereof for oral administration having an improved dissolution profile and an improved physical stability without affecting the release profile of said active ingredients. In addition, it is an aim of the present invention to provide a process for preparing said pharmaceutical compositions, particularly tablets, by means of a process that can be applied at an industrial level with low energy costs and which does not subject the active ingredient to aggressive formulation conditions which can entail the loss of stability of the product.

The authors of the present invention have found that the use of entacapone or a pharmaceutically acceptable salt thereof in the form of granules and their separate addition to levodopa and carbidopa or their pharmaceutically acceptable salts provides compositions wherein levodopa and carbidopa do not form part of entacapone's granules allowing the preparation of oral pharmaceutical compositions, particularly in the form of tablets, with an improved physical stability without affecting the dissolution properties of the oral compositions, making them suitable for therapeutic use.

It is worthwhile mentioning that the invention does not exclude compositions wherein some levodopa and/or carbidopa may be present onto the surface of the entacapone's granules as a result of the mixture between entacapone's granules and the rest of the active ingredients. It is clarified clear that this is not considered to be a situation wherein levodopa and carbidopa do form part of entacapone's granules.

Accordingly, a first aspect of the present invention is a solid pharmaceutical composition for oral intake comprising entacapone, levodopa and carbidopa or pharmaceutically acceptable salts or hydrates thereof characterized in that entacapone or a pharmaceutically acceptable salt thereof is in the form of granules and in that levodopa and carbidopa do not form part of entacapone's granules. Preferably more than 80% of entacapone or a pharmaceutically acceptable salt thereof in the solid oral pharmaceutical composition is separated from levodopa and carbidopa.

In a second aspect the present invention provides a solid oral pharmaceutical composition of entacapone, levodopa and carbidopa or pharmaceutically acceptable salts or hydrates thereof characterized in that entacapone or a pharmaceutically acceptable salt thereof is in the form of granules and it is added separately to levodopa and carbidopa.

In an embodiment, the pharmaceutical composition is in the form of a tablet. According to a particular embodiment, levodopa and carbidopa are together in the form of granules. In other embodiment, levodopa and carbidopa are in the form of powders.

In another embodiment, the pharmaceutical composition is in the form of a capsule. According to a particular embodiment, levodopa and carbidopa are together in the form of granules. In other embodiment, levodopa and carbidopa are in the form of powders.

In a particular embodiment, the entacapone granules are coated with a film coating formulation.

A third aspect of the present invention is a method for preparing a solid oral pharmaceutical composition according as defined above, comprising the stages of:
a) providing entacapone or a pharmaceutically acceptable salt thereof and, optionally mixing it with a disintegrant agent and/or a diluent agent and a binder;
b) granulating the product obtained in step a);
c) sieving the granules obtained in step b);
d) adding the granules obtained in step c) to a mixture of levodopa and carbidopa or pharmaceutically acceptable salts thereof optionally together with excipients.

### DETAILED DESCRIPTION OF THE INVENTION

In the present invention, “entacapone” is understood to be the compound entacapone or a pharmaceutically acceptable salt, hydrate o solvate thereof. Entacapone may be incorporated in the granules in any solid form either as a free compound or as a hydrate or solvate.

Unless otherwise indicated, as “levodopa” and “carbidopa” it is understood the compounds levodopa and carbidopa or their pharmaceutically acceptable salts, hydrates or solvates.

Entacapone, levodopa and carbidopa are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drugs substances are preferably above 50%, more preferably above 70%, most preferably above 90%. In a preferred embodiment it is above 95% of the drugs or of their salts, solvates, hydrates or prodrugs.

The invention provides a solid oral pharmaceutical composition of entacapone, levodopa and carbidopa or pharmaceutically acceptable salts thereof characterized in that entacapone or a pharmaceutically acceptable salt thereof is in the form of granules and it is added separately to levodopa and carbidopa.

By the term “granules” it is understood a core which comprises entacapone as the active ingredient and, which may optionally comprise at least one binder agent, one diluent agent and/or a disintegrant agent. In addition, the granules may optionally be coated with a film coating formulation.

The binder agent included in the core of the granules is selected from povidone, cornstarch, hydroxypropylcellulose, methyl cellulose, pregelatinized starch, low-substituted hydroxypropyl cellulose, copovidone and mixtures thereof.

The diluent agent optionally included in the core is preferably selected from microcrystalline cellulose, cellulose powdered, lactose monohydrate and dibasic calcium phosphate. Examples of diluents which can be used in the invention include, without limitation, saccharides such as monosaccharides, oligosaccharides or polysaccharides, and/or their oxidised and/or reduced forms; ribose, lactose in its various forms, anhydrous, monohydrate, agglomerated forms or atomised forms; sugar alcohols such as mannitol, maltol, sorbitol, maltitol, xylitol, isomalt and erythritol, cellulose powder, microcrystalline cellulose, silified microcrystalline cellulose or derivatives of cellulose modified chemically, such as hydroxypropyl cellulose, hydroxypropyl methyl cellulose; isomalt, starch, sucrose, pharmaceutically acceptable inorganic compounds such as dibasic calcium phosphate, carbonates of calcium or of magnesium, magnesium oxide, sugar alcohols selected from mannitol, sorbitol, maltitol, maltol, isomalt, xylitol, erythritol, or mixtures thereof.

The disintegrant agent optionally included in the core of the granule include natural starches, such as maize starch and potato starch; directly compressible starches such as starch 1500; modified or pregelatinized starches such as carboxymethylstarches and sodium starch glycolate; natural or chemically-modified cellulose, especially crosslinked sodium carboxymethyl cellulose (crossearmelose sodium) or low substituted hydroxypropyl cellulose; microcrystalline cellulose; gum, especially agar gum, and guar gum; alginic acid or salts thereof; acetates and citrates; sugars (especially lactose, mannitol and sorbitol); aluminum oxide; synthetic polymers such as cross-linked polyvinylpyrrolidones, specially crospovidone.

The film coating formulation used to coat the granule may comprise at least one polymer and/or one plasticizer or/and a colorant.

Accordingly, polymers usable in the film coating formulation are cellulose ethers, particularly, hydroxypropyl cellulose, hydroxypropyl methylcellulose, methylcellulose and mixtures thereof or, alternatively, acrylics, such as methacrylate and methyl methacrylate copolymers or vinyls such as polyvinyl alcohol. Examples of plasticizers usable in the film coating formulation are triethyl citrate, triacetin, propylene glycol, glycerolum, macrogolum, and mixtures thereof. Examples of colorants usable in the film coating formulation are titan dioxide, yellow and red iron oxides and the like.

The granule formulation described above can be prepared by any method known in the state of the art. However, in a particular embodiment, said granule formulation is prepared by wet granulation of entacapone optionally with a binder, a diluent agent and/or a disintegrant agent, followed by a coating process.

The composition of the invention can preferably be obtained by mixing entacapone granules with levodopa and carbidopa which may be granulated together or added extragranularly as such (in powder form) into the mixture to be formulated, e.g. into the tablet mass to be compressed or into the mass to be put into a capsule.

The solid oral pharmaceutical composition described above can be prepared by any method known in the art. However, in a particular embodiment, entacapone granules are prepared by wet granulation. Accordingly, the process for preparing such a composition comprises the steps of
a) providing entacapone or a pharmaceutically acceptable salt thereof and, optionally mixing it with at least one binder agent, one diluent agent and/or a disintegrant agent;
b) granulating the product obtained in step a);
c) sieving the granules obtained in step b)
d) adding the granules obtained in step c) to a mixture of levodopa and carbidopa or pharmaceutically acceptable salts thereof optionally together with excipients

The first step consists of providing entacapone or a pharmaceutically acceptable salt thereof and optionally mixing it with a binder, a disintegrant and/or a diluent.

The amount of entacapone in the granules is comprised between 50% and 95% of the total weight of the granule formulation, preferably between 65 and 90%.

The amount of disintegrant to be optionally added is comprised between 1% and 20% by weight with respect to the total weight of the granule formulation. Preferably, between 3% and 15% is added, more preferably between 5% and 12% of the total weight of the granule formulation. The amount of diluent to be optionally added is comprised between 10% and 40% by weight with respect to the total weight of the granule formulation, preferably between 15% and 20% of the total weight. The amount of binder to be optionally added to the mixture obtained in step a) is comprised between 1% and 15% by weight with respect to the total weight of the granule formulation.

In the second step, the granulation process may include the dry granulation or wet granulation. Wet granulation is preferred. In the wet granulation process, the solvent is added in an amount comprised between 10% and 50% by weight with respect to the weight of mixture to be granulated, which will serve to carry out the wet mixture. Alternatively, the binder agent can be previously dissolved or suspended in said solvent and then added to entacapone or to the mixture of entacapone and excipients. As solvents for preparing the wet mixture, water, hydroalcoholic mixtures and alcohols can be used, being preferred the use of water as a solvent for the granulation of the mixture. This solvent is later eliminated from the composition by means of a drying step.

Once the wet granules are obtained, they are subjected to a drying process to eliminate the solvent. Subsequently, the dried granules are calibrated by sieving or milling.

Optionally, the sieved or milled granules are coated with film coating solution. The coating process can be carried out by mixing the granules with the film coating formulation by any process known by a skilled person.

Finally, the entacapone granules thus obtained are added to carbidopa and levodopa together with appropriate excipients known by a skilled person in order to manufacture capsules or tablets. In a preferred embodiment, levodopa and carbidopa are added together in the form of granules.

Preferably, the formulation is in the form of a tablet. The tablet can be further coated. The coating process can be carried out by any process known by a skilled person.

The amount of levodopa in the formulation is comprised between 5% and 40% of the total weight of the formulation, preferably between 10 and 25%.

The amount of carbidopa in the formulation is comprised between 1% and 10% of the total weight of the formulation, preferably between 3 and 6%.

Additionally, the composition of the present invention can be either immediate release or sustained release.

By “immediate release” it is understood a release form in which greater than or equal to about 50% or more, preferably about 75% of the active ingredient is released within two hours of administration, preferably within one hour of administration.

By “sustained release” it is understood a release form in which the active ingredient is released at such a rate that blood (e.g. plasma) levels are maintained within a therapeutic range but below toxic levels for at least 8 hours, preferably at least about 12 hours after administration.

Suitable dose forms for oral administration may further contain conventional excipients known in the art such as binding agents, for example syrup, acacia, cellulose derivatives (i.e. hydroxypropylcellulose, carboxymethylcellulose, etc.) gelatin, sorbitol, tragacanth, or polyvinylpyrrolidone; fillers, for example lactose, sugar, maize starch, calcium phosphate, sorbitol or mannitol; tabletting lubricants, for example magnesium stearate; disintegrants, for example starch, crospovidone, sodium starch glycolate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agents throughout those compositions employing large quantities of fillers. Such operations are conventional in the art. The tablets may for example be prepared by wet or dry granulation and optionally coated according to methods well known in normal pharmaceutical practice, in particular with an enteric coating.

The mentioned formulations will be prepared using standard methods such as those described or referred to in the European and US Pharmacopoeias and similar reference texts.

In a preferred embodiment of the invention, the pharmaceutical composition is in the form of a tablet. The excipients used for preparing tablets may comprise between 0.5% and 4% by weight with respect to the total weight of the tablet of one or more lubricating agents, between 1% and 20% by weight of one or more disintegrants, between 5% and 50% by weight of one or more diluents and between 0.1% and 2% by weight of a glidant.

As disintegrant, sodium starch glycollate, low-substituted hydroxypropylcellulose, hydroxyethylcellulose, crospovidone, croscarmellose, starch, sodium carboxymethyl starch, casein derivatives or mixture thereof can be used.

As lubricating agent, magnesium stearate, calcium stearate, glyceryl palmitostearate, talcum, stearic acid, glyceryl behenate, sodium lauryl sulfate, sodium stearyl fumarate or mixtures thereof can be used. A stearate will preferably be used, still more preferably, magnesium stearate.

As diluent, a saccharide (monosaccharide or oligosaccharide, polysaccharides) and/or their oxidized and/or reduced forms; lactose in its anhydrous, monohydrate, agglomerated or spray forms; mannitol; cellulose powder, microcrystalline cellulose, silicified microcrystalline cellulose or chemically modified cellulose derivatives, such as hydroxypropylcellulose, hydroxypropylmethylcellulose; starch, sucrose, pharmaceutically acceptable inorganic compounds such as dibasic calcium phosphate, calcium or magnesium carbonates, magnesium oxide, or mixtures thereof can be used.

Additionally, previously prepared coprocessed diluents can be used such as Cellactose®, coprocessed lactose and cellulose powder, or Microcellac®, coprocessed lactose and microcrystalline cellulose, among others.

As glidant, anhydrous or hydrated colloidal silica, silicon dioxide colloidal anhydrous, magnesium trisilicate or talc can be used.

In the following, the present invention is further illustrated by examples. They should in no case be interpreted as a limitation of the scope of the invention as defined in the claims.

### EXAMPLES

### Example 1

| | mg | mg per | mg |
|---|---|---|---|
| | per tablet | tablet | per tablet |
| 1. Entacapone | 200 | 200 | 200 |
| 2. Povidone | 13 | 13 | 13 |
| 3. Crospovidone | 14 | 14 | 14 |
| 4. Microcrystalline cellulose | 50 | 50 | 50 |
| Entacapone granules total | 277 | 277 | 277 |
| 5. Levodopa | 50 | 100 | 150 |
| 6. Carbidopa monohydrate | 13.5 | 27 | 40.5 |
| 7. Povidone | 5 | 10 | 15 |
| 8. Crospovidone | 13 | 16 | 21 |
| 9. Microcrystalline cellulose | 25 | 45 | 80 |
| Levodopa/Carbidopa granules total | 106.5 | 198 | 306.5 |
| 10. Microcrystalline cellulose | 57 | 83 | 102 |
| 11. Silicon dioxide colloid. anhydrous | 2.5 | 3 | 4 |
| 12. Magnesium stearate | 7 | 9 | 10.5 |
| Total tablet weight | 450 | 570 | 700 |

### Example 2

| | mg | mg per | mg |
|---|---|---|---|
| | per tablet | tablet | per tablet |
| 1. Entacapone | 200 | 200 | 200 |
| 2. Pregelatinized starch | 28 | 28 | 28 |
| 3. Sodium starch glycollate | 13 | 13 | 13 |
| 4. Lactose granular | 50 | 50 | 50 |
| Entacapone granules total | 291 | 291 | 291 |
| 5. Levodopa | 50 | 100 | 150 |
| 6. Carbidopa monohydrate | 13.5 | 27 | 40.5 |
| 7. Pregelatinized starch | 12 | 22 | 30.5 |
| 8. Sodium starch glycollate | 12 | 17 | 22 |
| 9. Lactose granular | 25 | 45 | 80 |
| Levodopa/Carbidopa granules total | 112.5 | 211 | 323 |
| 10. Calcium hydrogen phosphate | 37 | 56 | 71.5 |
| 11. Silicon dioxide colloid. anhydrous | 2.5 | 3 | 4 |
| 12. Magnesium stearate | 7 | 9 | 10.5 |
| Total tablet weight | 450 | 570 | 700 |

### Example 3

| | mg per | mg per | mg per |
|---|---|---|---|
| | tablet | tablet | tablet |
| 1. Entacapone | 200 | 200 | 200 |
| 2. Low substituted hydroxypropyl cellulose | 28 | 28 | 28 |
| 3. Crospovidone | 13 | 13 | 13 |
| 4. Calcium hydrogen phosphate | 50 | 50 | 50 |
| Entacapone granules total | 291 | 291 | 291 |
| 5. Levodopa | 50 | 100 | 150 |
| 6. Carbidopa monohydrate | 13.5 | 27 | 40.5 |
| 7. Low substituted hydroxypropyl cellulose | 12 | 22 | 30 |
| 8. Crospovidone | 12 | 17 | 22.5 |
| 9. Calcium hydrogen phosphate | 25 | 45 | 80 |
| Levodopa/Carbidopa granules total | 112.5 | 211 | 323 |
| 10. Calcium hydrogen phosphate | 37 | 56 | 71.5 |
| 11. Silicon dioxide colloid. anhydrous | 2.5 | 3 | 4 |
| 12. Magnesium stearate | 7 | 9 | 10.5 |
| Total tablet weight | 450 | 570 | 700 |

### Manufacturing Method

The tablets of the above examples are obtained according to the following procedure:
a) component 1 is mixed with compounds 2, 3 and 4. The mixture is granulated in a suitable mixer with purified water, dried and sieved.
b) components 5 and 6 are mixed with compounds 7, 8 and 9. The mixture is granulated in a suitable mixer with purified water, dried and sieved.
c) mix the granules obtained in both steps a) and b) together with compounds 10 and 11
d) add compound 12 to the mixture obtained in step c)
e) the mixture of step d) is compressed in a tabletting machine equipped with suitable punches
f) tablets are coated with colored HPMC water solution to the weight gain of 2-3%

### Example 4

| | mg | mg per | mg |
|---|---|---|---|
| | per tablet | tablet | per tablet |
| 1. Entacapone | 200 | 200 | 200 |
| 2. Methyl cellulose | 19 | 19 | 19 |
| 3. Sodium starch glycollate | 13 | 13 | 13 |
| 4. Lactose granular | 50 | 50 | 50 |
| Entacapone granules total | 282 | 282 | 282 |
| 5. Levodopa | 50 | 100 | 150 |
| 6. Carbidopa monohydrate | 13.5 | 27 | 40.5 |
| 7. Methyl cellulose | 8 | 15 | 23 |
| 8. Sodium starch glycollate | 12 | 17 | 22 |
| 9. Lactose granular | 25 | 45 | 80 |
| Levodopa/Carbidopa granules total | 108.5 | 204 | 315.5 |
| 10. Lactose granular | 50 | 72 | 88 |
| 11. Silicon dioxide colloid. anhydrous | 2.5 | 3 | 4 |
| 12. Magnesium stearate | 7 | 9 | 10.5 |
| Total tablet weight | 450 | 570 | 700 |

### Example 5

| | mg | mg per | mg |
|---|---|---|---|
| | per tablet | tablet | per tablet |
| 1. Entacapone | 200 | 200 | 200 |
| 2. Methyl cellulose | 19 | 19 | 19 |
| 3. Crospovidone | 13 | 13 | 13 |
| 4. Calcium hydrogen phosphate | 50 | 50 | 50 |
| Entacapone granules total | 282 | 282 | 282 |
| 5. Levodopa | 50 | 100 | 150 |
| 6. Carbidopa monohydrate | 13.5 | 27 | 40.5 |
| 7. Methyl cellulose | 8 | 15 | 23 |
| 8. Sodium starch glycollate | 12 | 17 | 22 |
| 9. Calcium hydrogen phosphate | 25 | 45 | 80 |
| Levodopa/Carbidopa granules total | 108.5 | 204 | 315.5 |
| 10. Calcium hydrogen phosphate | 50 | 72 | 88 |
| 11. Silicon dioxide colloid. anhydrous | 2.5 | 3 | 4 |
| 12. Magnesium stearate | 7 | 9 | 10.5 |
| Total tablet weight | 450 | 570 | 700 |

### Manufacturing Method

The tablets of examples 4 and 5 are obtained according to the following procedure:
a) component 1 is mixed with compounds 3 and 4. Dissolve compound 2 in water and granulate the mixture in a suitable mixer with solution of methyl cellulose in water, dried and sieved.
b) components 5 and 6 are mixed with compounds 7, 8 and 9. The mixture is granulated in a suitable mixer with solution of methyl cellulose in water, dried and sieved.
c) mix the granules obtained in both steps a) and b) together with compounds 10 and 11
d) add compound 12 to the mixture obtained in step c)
e) the mixture of step d) is compressed in a tabletting machine equipped with suitable punches
f) tablets are coated with colored HPMC water solution to the weight gain of 2-3%

### Example 6

| | mg per | mg per | mg per |
|---|---|---|---|
| | tablet | tablet | tablet |
| 1. Entacapone | 200 | 200 | 200 |
| 2. Low substituted hydroxypropyl cellulose | 25 | 25 | 25 |
| 3. Crospovidone | 12.5 | 12.5 | 12.5 |
| 4. Microcrystalline cellulose | 50 | 50 | 50 |
| Entacapone granules total | 287.5 | 287.5 | 287.5 |
| 5. Levodopa | 50 | 100 | 150 |
| 6. Carbidopa monohydrate | 13.5 | 27 | 40.5 |
| 7. Crospovidone | 12.5 | 17.5 | 22.5 |
| 8. Microcrystalline cellulose | 77 | 126 | 185 |
| 9. Silicon dioxide colloid. anhydrous | 2.5 | 3 | 4 |
| 10. Magnesium stearate | 7 | 9 | 10.5 |
| Total tablet weight | 450 | 570 | 700 |

### Manufacturing Method

The tablets of the above example are obtained according to the following procedure:
a) component 1 is mixed with compounds 3 and 4. Dissolve compound 2 in water and granulate the mixture in a suitable mixer with solution of methyl cellulose in water, dried and sieved.
b) components 5, 6, 7, 8 and 9 are homogeneously mixed with the granules obtained in step a).
c) and compound 10 to the mixture powder obtained in step b) is compressed in a tabletting machine equipped with suitable punches

1. A solid pharmaceutical composition for oral intake comprising entacapone, levodopa and carbidopa or pharmaceutically acceptable salts or hydrates thereof characterized in that entacapone or a pharmaceutically acceptable salt thereof is in the form of granules and in that levodopa and carbidopa do not form part of entacapone's granules.

2. The solid oral pharmaceutical composition of claim 1 wherein more than 80% of entacapone or a pharmaceutically acceptable salt thereof is separated from levodopa and carbidopa.

3. The solid oral pharmaceutical composition of claim 1 wherein levodopa and carbidopa are together in the form of granules.

4. The solid oral pharmaceutical composition of claim 1 wherein levodopa and carbidopa are together in the form of powders.

5. The solid oral pharmaceutical composition of claim 1 in the form of a capsule.

6. The solid oral pharmaceutical composition of claim 1 in the form of a tablet.

7. The solid oral pharmaceutical composition of claim 1 wherein the entacapone granules are coated with a film coating formulation.

8. A method for preparing a solid oral pharmaceutical composition according to claim 1, comprising the steps of:
a) providing entacapone or a pharmaceutically acceptable salt thereof and, optionally mixing it with at least one binder agent, one diluent agent and/or a disintegrant agent;
b) granulating the product obtained in step a);
c) sieving the granules obtained in step b);
d) adding the granules obtained in step c) to a mixture of levodopa and carbidopa or pharmaceutically acceptable salts thereof, optionally together with excipients.

9. The method of claim 8 wherein levodopa and carbidopa in step d) are added together in the form of granules.
